(19) United States
(12) Patent Application Publication
(10) Pub. No.: US 2010/0086524 A1
(43) Pub. Date: Apr. 8, 2010

(54) CELLULAR PREPARATIONS FOR USE AS A REVASCULARIZATION STIMULATING AGENT

(76) Inventors: Gérard Tobelem, Paris (FR); Sophie Le Ricousse-Roussanne, Champigny-sue-Marne (FR); Philippe Foubert, La Garde (FR); Jean-Sébastien Silvestre, Sceaux (FR)

Correspondence Address: OSTROLENK FABER GERB & SOFFEN, 1180 AVENUE OF THE AMERICAS, NEW YORK, NY, 100368403, US

(21) Appl. No.: 12/517,281

(22) PCT Filed: Dec. 6, 2007
(86) PCT No.: PCT/FR07/02003

§ 371 Date: Jul. 15, 2009

(30) Foreign Application Priority Data
Dec. 6, 2006 (FR) 0610638

(51) Int. Cl. A61K 35/12; A61P 17/02

(52) U.S. Cl. 424/93.7

(57) ABSTRACT

The invention relates to a cellular preparation containing endothelial cell precursors (EPCs) and smooth muscular cell precursors (SMCs), as a combination product for simultaneous, separated or time-spread administration, used as a revascularisation stimulating agent. The invention also relates to the use of such a cellular preparation.

The present invention relates to a cell preparation and to the use of that cell preparation as an agent stimulating revascularization.

A large number of diseases are targeted here, and overall are diseases which have led to denaturation or destruction of a vascular system. Such circumstances are in particular encountered when the arterial blood supply into a tissue or an organ decreases or ceases.

Cell therapies consist precisely in regenerating a degraded tissue, whatever it may be, on the basis of specific cells cultured in vitro, or otherwise, and then transplanted into the degraded tissue. Many advances have already been made for the treatment of different diseases by means of these cell therapies.

The general principle essentially relies on the ability which certain types of cells have at certain stages of their development, to multiply and differentiate to produce specialized cells which acquire a morphology and a specific function of the tissue into which they are implanted. Embryonic stem cells, in the initial stages after fertilization, are undifferentiated and will be able to lead to the formation of all the tissues of the body. Conversely adult stem cells are already engaged in a specific tissue program and they can only lead to the formation or the regeneration of distinct tissues; they are referred to as multipotent whereas the embryonic stem cells are totipotent. Moreover, the precursor cells derived from division of stem cells have already during their development acquired a certain degree of specialization and are physiologically functional.

Thus, the idea of preparing populations of multipotent stem cells on the basis of tissues from skeletal muscle has already been proposed. Reference can in particular be made to the document WO03/027281 which describes a process of preparation of such multipotent stem cells capable of differentiating into skeletal muscle cells and into many other cells, and in particular into smooth muscle cells, into cardiomyocytes, into blood cells, into vascular endothelial cells, into adipocytes, etc.

This process makes it possible to prepare populations of multipotent stem cells with a view to the regeneration of many types of tissue, on the other hand it is relatively complex to put into practice.

Moreover, since the tissues of the myocardium are devoid of stem cells capable of forming cardiomyocytes in order to regenerate, the idea of preparing relatively homogenous cell populations wherein the dominant type has the characteristics of myoblastic cells, and injecting these populations directly into the myocardial tissue or else indirectly into the arterial circulation has also been suggested. Reference can also be made to the document WO01/94555, wherein such a procedure is described.

There also, the sorting of the different cell populations requires the observation of specific cell markers. Moreover, the cell populations obtained are specifically suited to the regeneration of the cardiac muscle.

Thus the first document analyzed above presents a relatively wide use spectrum of the populations of multipotent stem cells obtained, which does not however make it possible to obtain convincing results for pro-angiogenic activity, whereas the second document discloses specific cell populations exhibiting the characteristics of myoblastic cells and consequently having more restricted uses.

Also, one problem which arises and which the present invention aims to solve is to provide a cell preparation for use as an agent stimulating revascularization which makes it possible to reconstitute the vascular system of damaged mammalian, and in particular human, tissue when it is administered. Further, it is necessary to be able to obtain such a preparation relatively easily.

With the aim of solving this problem, the present invention proposes a cell preparation containing precursors of endothelial cells (EPCs, for Endothelial Precursor Cells) and precursors of smooth muscle cells (SMCs, for Smooth Muscle Cells), as a combination product for simultaneous, separate, or staggered administration, for use as an agent stimulating revascularization.

Thus, one characteristic of the invention consists in the utilization of two types of cells, both endothelial precursor cells and smooth muscle cell precursors which then interact to stimulate the formation and the development of blood capillaries on the basis of preexisting blood vessels. Thus, from any cell tissue whatever, in which the blood circulation had been decreased or stopped, the injection of endothelial precursor cells and smooth muscle cell precursors into the infarcted tissue thus induces the regeneration of the blood capillaries, on condition of course that the blood circulation reappears normally.

Said endothelial precursor cells (EPCs) are preferably obtained by in vitro differentiation of progenitors deriving from umbilical cord blood or from hematopoietic bone marrow or else from peripheral circulating blood or indeed from any other tissue. As will be explained in more detail later, mononuclear cells are isolated, for example from the umbilical cord blood then in vitro differentiation thereof is induced, in order then to recover them and combine them with the smooth muscle precursor cells.

Particularly advantageously, said endothelial precursor cells (EPCs) express a specific receptor capable of accepting a protein material so as to activate said endothelial precursor cells (EPCs). Thus the activation of endothelial precursor cells induces a supplementary synergy between these precursor cells and the smooth muscle cell precursors, a synergy which then leads to revascularization or pro-angiogenic activity greater than that of the non-activated precursor cells alone or of the smooth muscle cell precursors alone.

Advantageously, said specific receptor is an Eph receptor with tyrosine kinase activity, for example of the EphB type, and more precisely EphB4. In addition, the protein material preferably contains a specific ligand of said marker, said ligand being associated with a binding polypeptide. Further, and according to a preferred implementation mode, the specific ligand is an ephrine ligand, for example ephrine B or more precisely ephrine B2 or else ephrine B1. As regards the binding polypeptide, this is preferably an Fc immunoglobulin fragment.

In this manner, endothelial cells which exhibit specific receptors, with which specific receptors a protein material consisting of a ligand and a binding polypeptide is combined are obtained; such endothelial precursor cells are then activated.

As regards the smooth muscle cell precursors (SMCs), these are preferably obtained by in vitro differentiation of progenitors deriving from umbilical cord blood or from hematopoietic bone marrow or else from peripheral circulating blood or indeed from any other tissue. Just as with the endothelial precursor cells, mononuclear cells are first isolated, for example from umbilical cord blood and then the differentiation thereof into smooth muscle cell precursors is induced. They are then recovered, and then combined with the endothelial precursor cells for administration.

According to another implementation mode of the invention, said smooth muscle cell precursors (SMCs) are obtained from a biopsy of muscle tissue which is taken from a skeletal muscle of a human being and which is placed in culture so as to harvest only the smooth muscle cell precursors identified. These smooth muscle cell precursors are in fact clearly identifiable on account of specific cell markers.

According to another aspect, the present invention proposes the use of a cell preparation combining endothelial precursor cells and smooth muscle cell precursors for the preparation of a cell composition intended to stimulate the revascularization or angiogenesis of ischemic tissues in mammals and in particular in man. In addition, a combination of these endothelial precursor cells and smooth muscle cell precursors for the preparation of a medicament intended to normalize tumor revascularization is also envisaged.

More broadly, the use of a cell preparation combining endothelial precursor cells and smooth muscle cell precursors for the preparation of a medicament intended to stimulate the revascularization of damaged tissues in the healing phase is envisaged. Thus, the treatment of pathological conditions such as: radiation or post-radiation dermatitis, burns or skin damage following trauma or of any other origin with the aid of the cell preparation according to the invention is envisaged.

In addition, the cell preparation combining endothelial precursor cells and smooth muscle cell precursors is suitable for the preparation of a therapeutic composition intended for the prevention or the treatment of cancers with administration prior to or simultaneous with anti-cancer treatment by chemotherapy or by radiotherapy. It is also suitable for treating vascular malformations and in particular angiomas.

More generally, the use of a cell preparation of the type according to the invention as a pro-angiogenic active ingredient, in combination with a physiologically acceptable excipient, for the preparation of a composition for therapeutic use in the treatment of vascular insufficiency, in particular in the revascularization of cardiac, cerebral or peripheral ischemic tissues is envisaged.

Other features and advantages of the invention will emerge from the reading of the description given below of particular modes of implementation of the invention, given for information, but without limitation, with reference to sole appended diagram this being:

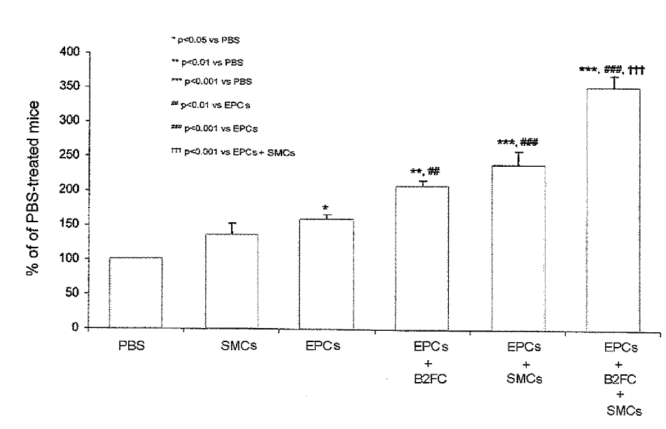

FIG. 1, which is a histogram showing a comparison of the efficacy of the pro-angiogenic activity of the cell preparations which are the subject of the present invention.

### FIRST IMPLEMENTATION MODE

Thus, according to a first implementation mode of the invention, the endothelial precursor cells (EPCs) and the smooth muscle cell precursors (SMCs) are jointly obtained from human umbilical cord blood.

Also, according a first preparation mode, samples of human umbilical cord blood, each of 30 to 50 ml, are firstly collected and these are placed in sterile tubes containing an anticoagulant solution of sodium heparin. The mononuclear cells are next isolated from the umbilical cord blood by density gradient centrifugation by means of Pancoll (1.077 g par milliliter, product marketed by Dominique Dutscher S. A., Brumath, France). The mononuclear cells isolated are next separated from adhering cells by culturing on plastic boxes for 24 hours at 37° C. A mixture of mononuclear cells isolated and freed from adhering cells is then recovered, and the mixture is next placed in the wells of a plate with six wells covered with a defined matrix. This defined matrix contains fibronectin, laminin, sodium heparan sulfate, type I and type IV collagen (these products are all supplied by Sigma-Aldrich) and a growth factor hVEGF (R&D Systems, Oxford UK).

In this manner, after 15 days of culturing, the appearance of colonies of squamous type and colonies of fusiform type is clearly discerned. Each of these colonies is then recovered in order to prick them out again independently of one another, then to amplify them in order to obtain large quantities of cells of these two types.

The squamous type cells are in fact endothelial precursor cells since they express the principal markers of this type of cells, Von Willebrand Factor, CD31, eNOS, VE-Cadherine, VEGF-R1 or VEGF-R2. And the fusiform type cells are smooth muscle cell precursors since as markers they express αSMA, calponin, SM22α and SM-MHC.

A first cell preparation which thus simultaneously contains endothelial precursor cells and the smooth muscle cell precursors is tested on batches of Nude male mice according to a first protocol defined below.

The efficacy of the aforesaid preparation will be compared, not only in relation to a neutral control preparation with PBS (buffer: 137 mM NaCl, 2.7 mM KCl, 10 mM Na₂HPO₄, 1.84 mM KH₂PO₄) devoid of cell colonies, but also in relation to a preparation containing only endothelial precursor cells and to a preparation containing only smooth muscle cell precursors. Hence four batches of six animals will be reserved, and the four preparations will be administered respectively to the animals of the four batches.

First of all, at time t=0, ligature of the right femoral artery of all the mice aged seven weeks is performed in order to simulate ischemia. Five hours later, the first cell preparation of endothelial precursor cells and of smooth muscle cell precursors are injected simultaneously by the intravenous route at the level of the retro-orbital sinus, at the rate of about 250,000 cells for the two types of cells and for each mouse of a first batch, and the other preparations respectively to the other three batches of animals. The other cell preparations tested will contain about 500,000 cells.

Next, 12 days after the ligature, the mice are sacrificed and the gastronecmius muscles of the ischemized leg and of the non-ischemized leg are removed. Three parameters are then measured, the angiography score, the capillary density and the cutaneous blood flow.

The angiography score measures the density of the vessels and it is determined by micro-angiography (operating procedures in the article by Silvestre J. S et al., Cir. Res., 2001; 89: 259-264). In this particular case, the density of the vessels is measured for each animal in the ischemized limb and in the non-ischemized limb and the result obtained is presented in the form of a ratio, ischemized leg divided by non-ischemized leg.

The capillary density, which represents the number of capillaries per square millimeter, is measured by labeling sections of the gastronecmius muscle by means of an antibody directed against the marker CD31, specific for the endothelial cells as indicated above, and by comparing these sections thereof with sections of the same muscle from the non-ischemized limb. The results thus obtained are presented in the form of the ratio, ischemized leg divided by non-ischemized leg.

As for the cutaneous blood flow, this is evaluated quantitatively by the ratio of the blood flow measured on the ischemized limb and the blood flow measured on the non-ischemized limb. This checks that the change in the number of vessels corresponds to a functional adaptation and hence to a change in the perfusion of the ischemized limb.

### Results Table I

The measurement of the aforesaid three parameters is set out in the following table for each of the four groups of six individuals. The measurement is the result of the mean taken over the six animals.

| | PBS | | | |
|---|---|---|---|---|
| | Control | EPCs | SMCs | SMCs + EPCs |
| Angiography score | 100 | 157 ± 8 | 134 ± 18 | 239 ± 20 |
| Capillary density | 100 | 163 ± 9 | 144 ± 6 | 226 ± 16 |
| Cutaneous blood flow | 100 | 150 ± 6 | 141 ± 5 | 217 ± 18 |

Thus it is observed that the injection of endothelial precursor cells EPCs or of smooth muscle cell precursors SMCs, causes a slight increase, of about 60% and 35% respectively, in the density of the vessels in comparison to that of the “control” group of animals, to which the control PBS was administered (or respectively multiplied by 1.60 and 1.35). Unexpectedly, when the endothelial precursor cells and the smooth muscle cell precursors are injected simultaneously, the density of vessels is essentially multiplied by 2.4 in comparison to that of the “control” group of animals.

As regards the capillary density, it is observed that the animals to which the endothelial precursor cells and the smooth muscle cell precursors were administered simultaneously exhibit a capillary density essentially greater by 50% than the capillary density of the animals to which either endothelial precursor cells alone, or smooth muscle cell precursors alone were administered.

As for the measurement of the cutaneous blood flow of the four groups of animals, this corroborates the above results, since the cutaneous blood flow of the animals to which the endothelial precursor cells and the smooth muscle cell precursors were administered simultaneously is about 50% higher than the blood flow of the animals to which only the endothelial precursor cells or only the smooth muscle cell precursors were administered.

Thus the results for the three parameters measured, the angiography score, the capillary density and the cutaneous blood flow, which represent a measure of the angiogenic activity or of revascularization, appear to be in agreement and moreover in comparable proportions. Hence the combined action of the endothelial precursor cells and smooth muscle cell precursors within a previously ischemized tissue appears to induce regeneration of the vessels and the blood capillaries. Hence, a synergy of the endothelial precursor cells and the smooth muscle cell precursors due to a potentiation effect within the ischemized tissue induces this regeneration of the blood vessels.

Thus a cell preparation according to the invention incorporating endothelial precursor cells and smooth muscle cell precursors can be administered as a medicament, particularly for treating vascular insufficiency, in particular in the revascularization of ischemic cardiac, cerebral or peripheral tissues. Moreover, such a preparation is also indicated for normalizing tumor vascularization and thus enabling the drugs administered in chemotherapy to disperse better in the tumor. In fact, tumor vascularization bears little resemblance to the normal vascularization of healthy tissues and in particular is accompanied by hemostatic and chemical disorders. Thus the drugs administered reach the core of the tumor with greater difficulty which by the same token decreases their activity and their efficacy. Now, the aforesaid cell preparation has the effect of restoring normal vascularization to the tumor and consequently to its receiving normally the drugs circulating in the blood.

The cell preparation according to the invention can be packed in the form of a unit dose simultaneously containing the smooth muscle cell precursors and the endothelial precursor cells or else in the form of separate doses. In that case, the two cell compositions are administered, either simultaneously, or separately, or else staggered.

Moreover, the endothelial precursor cells and the smooth muscle cell precursors can be independently frozen and stocked at minus 80° C. Thus, when necessary, they are next thawed then cultured for a period of about one week for administration simultaneously or separately.

The cell preparation is in particular suitable for the preparation of a composition intended for the treatment of arteritis, coronary vascular or cardiac insufficiency and cerebral vascular insufficiency. In the aforesaid animal model, it gave good results in the treatment of so-called critical ischemia of the lower limbs, and hence there are great hopes of being able to obtain a cure in man under similar circumstances, making it possible to avoid amputation.

The cell composition according to the invention can thus be administered in mammals and in particular in man according to a procedure known per se. For example, the cell composition can be injected at or in the vicinity of the vascular lesion, into the blood, or else introduced directly at the site of the lesion by means of an appropriate vector. As a variant, it is possible to administer the endothelial precursor cells, on the one hand, and the smooth muscle cell precursors, on the other separately, by the injectable route.

In an adult human, a cell composition according to the invention containing between 10⁵ and 10⁹ cells can be administered by injection.

### SECOND IMPLEMENTATION MODE

According to a second implementation mode of the invention, activated endothelial precursor cells EPCs and smooth muscle cell precursors SMCs are combined.

In order to do this, endothelial precursor cells exhibiting a specific cell marker on the surface of the external membrane of the cell are provided, said cell marker being selected from the group consisting of the Eph, in particular EphB4 or EphB1, and a protein material of structure L-K is combined therewith. The protein material is made up of a specific ligand (L) of said marker, and of a binding peptide (K), in particular an Fc immunoglobulin or antibody fragment. The whole forming a system capable of providing cell populations or a cell material of structure EPC-Eph-L-K. Reference may be made to the document FR 05 08029 wherein the utilization of such a cell material is described.

Thus, in order to stimulate angiogenesis, the EPC cells containing the Eph marker are activated by the specific ligand L belonging to the ephrine family. The ligand L can also consist of a peptide fragment of an ephrine, for example of ephrine B2 which would then have the same biological activity.

In that case it is these active endothelial precursor cells which will be administered in combination with the smooth muscle cell precursors in order to stimulate the revascularization or angiogenesis of a previously ischemized tissue.

Preferably, according to this second implementation mode of the invention, the endothelial precursor cells are activated via a cell marker EphB4 to which an ephrine B2 ligand becomes attached, the ligand itself being combined with an Fc antibody fragment. Activated endothelial precursor cells of the form: EPC-EphB4-ephrine B2-Fc are thus obtained.

First of all, it is necessary to obtain a cell population mainly containing endothelial precursor cells exhibiting the marker EphB4.

According to a first obtention mode, cell colonies of the squamous type derived from the cell preparation obtained according to the aforesaid first preparation mode are recovered. This cell preparation inter alia contains endothelial precursor cells equipped with the marker EphB4. These cell colonies are then treated with 3 μg/ml of ephrine-B2-Fc fusion protein for an incubation period of 30 minutes at 37° C. Each non-bonded fusion protein is removed by rinsing with PBS (at least two rinsings are necessary). A composition of endothelial precursor cells of structure EPC-EphB4-ephrine B2-Fc is thus obtained.

Next, it is necessary to combine this cell composition with smooth muscle cell precursors in order to make up a second cell preparation according to the invention. The aforesaid cell preparation obtained according to the first preparation mode contains smooth muscle cell precursors identifiable by their fusiform shape. Hence, these smooth muscle cell precursors will be selected in such a preparation, and they will be combined with the endothelial precursor cells of structure EPC-EphB4-ephrine 2-Fc.

According to a second obtention mode, a cell composition is recovered at the end of the first part of the first preparation mode aforesaid in the first implementation mode of the invention. Thus the mono-nuclear cells from umbilical cord blood were isolated by centrifugation and these were then separated from adhering cells by culturing on plastic boxes for 24 hours at 37° C. A cell mixture containing mononuclear cells expressing the marker EphB4 and mononuclear cells not expressing said marker are then obtained.

Next, the CD34⁺ labeled cells are isolated and purified from the non-adhering cells by a standard immuno-magnetic separation technique, in particular by means of the “CD34 isolation Kit” (marketed by MILTENYI BIOTECH, Paris France), which contains an anti-CD34 monoclonal antibody. The analysis of the cells thus obtained, by flow cytometry and utilizing an anti-CD34 monoclonal antibody (preferably different from the former) coupled with FITC, shows that 75% (±5.6%) of them possess the CD34 marker.

The cell mixture thus obtained, which contains 1.5×10⁶ to 3.5×10⁶ CD34⁺ cells, can be placed into the wells of a 6-well plate covered with a matrix containing fibronectin, laminin, sodium heparan sulfate, and type I and IV collagen (products marketed by the aforesaid SIGMA-ALDRICH) and in a culture medium containing hVEGF, bFGF and IGF1 (products marketed by the company called R&D SYSTEMS INC., Oxford, United Kingdom). After 15 days' culturing, a cell mixture enriched in EPC-EphB4 is recovered.

This cell mixture is then treated in an identical way to the first obtention mode, with 3 μg/ml of ephrine-B2-Fc fusion protein. And a cell composition mainly comprising endothelial precursor cells of structure EPC-EphB4-ephrine B2-Fc is then obtained.

Next, smooth muscle cell precursors selected analogously to the first obtention mode are combined with this cell composition, in a cell preparation according to the first preparation mode. A third cell preparation according to the invention will thus be obtained.

It will also be noted that the smooth muscle cell precursors can also be obtained according to the aforesaid second obtention mode.

The second cell preparation simultaneously containing endothelial precursor cells of EPC-EphB4-ephrine B2-Fc structure and smooth muscle cell precursors is tested according to a second protocol essentially identical to the first protocol above, on batches of male Nude mice. It will be noted that the aforesaid third cell preparation would lead to the same result as the second preparation.

The efficacy of the second preparation will be compared in relation to a control preparation (PBS), and also in relation to a preparation containing only endothelial precursor cells of EPC-EphB4-ephrine B2-Fc structure. Three batches of six animals will thus be reserved, and the three preparations will be administered respectively to the animals of these three batches.

According to this second protocol, and similarly to the first, at a time t=0 ligature of the right femoral artery of all the mice aged seven weeks is effected in order to simulate ischemia. Five hours later, the second cell preparation at a rate of 250,000 cells for the two types of cells and for each mouse of a first batch, and the aforesaid preparations by way of comparison respectively to the animals of the two other batches, are injected simultaneously by the intravenous route at the level of the retro-orbital sinus.

Next, 12 days after the ligature, the mice are sacrificed and the gastronecmius muscles of the ischemized leg and of the non-ischemized leg are removed. The three parameters already encountered above, the angiography score, the capillary density and the cutaneous blood flow are then measured.

### Results Table II

The measurement of the aforesaid three parameters is set out in the following table for each of the three groups of six individuals. The measurement is the mean of the results observed in the six animals.

| | PBS | Ephrine-B2-Fc | SMCs + EphB4 |
|---|---|---|---|
| | Control | EPCs | ephrine-B2-Fc EPCs |
| Angiography score | 100 | 206 ± 10 | 350 ± 20 |
| Capillary density | 100 | 219 ± 16 | 259 ± 13 |

Thus, quite surprisingly it is observed that the endothelial precursor cells activated via their EphB4 marker linked to the ephrine B2 and to the antibody fragment Fc, and in combination with smooth muscle cell precursors, induce an increase in the density of the vessels equivalent to 3.5 times the density of the vessels obtained with the control composition which is devoid of cells.

What is more, the cell composition containing only endothelial cells activated by means of their EphB4 marker combined with the ligand ephrine B2 and with the Fc antibody fragment already induces an increase in the density of the vessels twice as great as that of the control composition.

Thus, the second cell preparation according to the invention incorporating activated endothelial precursor cells and in combination, smooth muscle cell precursors, can be administered as a medicament in order also to treat vascular insufficiency, in particular in the revascularization of cardiac, cerebral or peripheral ischemic tissues. Just like the first cell preparation, this second preparation is suitable for the creation of a medicament for normalizing tumor vascularization.

Reference will then be made to FIG. 1, on which the angiography scores of the cell preparations tested according to the invention are reproduced in the form of a histogram.

Thus, if it is noted that the progenitors of endothelial cells and the smooth muscle cell precursors independently produce a pro-angiogenic effect, it is remarkable that the combined action of these two cell types has a marked pro-angiogenic effect. The density of the vessels is in fact multiplied by 2.4 relative to the density of the vessels of the control composition.

In addition, it is equally remarkable that the combination of smooth muscle cell precursors and endothelial precursor cells activated via their EphB4 receptor, multiplies the density of the blood vessels by 3.5 relative to the density of vessels obtained with the control composition.

1. A cell preparation comprising endothelial precursor cells (EPCs) and smooth muscle cell precursors (SMCs), as a combination product for simultaneous, separate, or staggered administration, for use as a revascularization stimulating agent.

2. The cell preparation as claimed in claim 1, wherein said endothelial precursor cells (EPCs) are obtained by in vitro differentiation of progenitors deriving from umbilical cord blood.

3. The cell preparation as claimed in claim 1, wherein said endothelial precursor cells (EPCs) are obtained by in vitro differentiation of progenitors deriving from circulating blood.

4. The cell preparation as claimed in claim 1, wherein said endothelial precursor cells (EPCs) are obtained by in vitro differentiation of progenitors deriving from hematopoietic bone marrow.

5. The cell preparation as claimed in claim 1, wherein said endothelial precursor cells (EPCs) express a specific Eph receptor with tyrosine kinase activity capable of accepting a protein material to activate said endothelial precursor cells (EPCs).

6. The cell preparation as claimed in claim 5, wherein said specific receptor is a receptor of the EphB type.

7. The cell preparation as claimed in claim 6, wherein said specific receptor is a receptor of the EphB4 type.

8. The cell preparation as claimed in claim 5, wherein said protein material contains a specific ligand of said marker, said ligand being combined with a binding polypeptide.

9. The cell preparation as claimed in claim 8, wherein said specific ligand of said marker is an ephrine ligand.

10. The cell preparation as claimed in claim 9, wherein said specific ligand of said marker is an ephrine B ligand.

11. The cell preparation as claimed in claim 10, wherein said specific ligand of said marker is an ephrine B2 ligand.

12. The cell preparation as claimed in claim 8, wherein said binding polypeptide is an Fc immunoglobulin fragment.

13. The cell preparation as claimed in claim 1, wherein said smooth muscle cell precursors (SMCs) are obtained by in vitro differentiation of progenitors deriving from umbilical cord blood or from peripheral blood.

14. The cell preparation as claimed in claim 1, wherein said smooth muscle cell precursors (SMCs) are obtained by in vitro differentiation of progenitors deriving from hemato-poietic bone marrow.

15. The cell preparation as claimed in claim 1, wherein said smooth muscle cell precursors (SMCs) are obtained from a muscle biopsy.

16. The use of a cell preparation according to claim 1 for the preparation of a medicament intended to stimulate the revascularization of ischemic tissues.

17. The use of a cell preparation as claimed in claim 1 for the preparation of a medicament intended to normalize tumor vascularization in order to allow the drugs administered in chemo-therapy to spread into a tumor.

18. The use of a cell preparation as claimed in claim 1 for the preparation of a medicament intended to stimulate the revascularization of damaged tissues in the healing phase.
